(19) United States
(12) Patent Application Publication
(10) Pub. No.: US 2024/0183850 A1
(43) Pub. Date: Jun. 6, 2024

(54) REPLICA OF BIOLOGICAL MATERIAL AND METHOD FOR PRODUCING REPLICA OF BIOLOGICAL MATERIAL

(71) Applicant: SEIKO EPSON CORPORATION, TOKYO (JP)

(72) Inventors: Chikara KOJIMA, Matsumoto-shi (JP); Masamitsu MOCHIZUKI, CHINO-SHI (JP); Shinji TERAO, FUJIMI-MACHI (JP); Keita KUBO, Matsumoto-shi (JP); Kazuki Sano, MATSUMOTO-SHI (JP); Kiyoaki MURAI, MATSUMOTO-SHI (JP); Masahiko KOBAYASHI, CHINO-SHI (JP)

(21) Appl. No.: 18/530,252

(22) Filed: Dec. 6, 2023

(30) Foreign Application Priority Data
Dec. 6, 2022 (JP) 2022-195092

(51) Int. Cl. G01N 33/551

(52) U.S. Cl. CPC G01N 33/551

(57) ABSTRACT

Provided is a replica of a biological material, which has a three-dimensional structure similar to a three-dimensional structure of the biological material and has a surface made of an inorganic material. The inorganic material may be an oxide or a nitride. The replica may include a body and a coating film which covers a surface of the body and is made of the inorganic material.

The present application is based on, and claims priority from JP Application Serial Number 2022-195092, filed Dec. 6, 2022, the disclosure of which is hereby incorporated by reference herein in its entirety.

### BACKGROUND

### 1. Technical Field

The present disclosure relates to a replica of a biological material and a method for producing the replica of a biological material.

### 2. Related Art

In the fields of food hygiene, medical diagnosis, environmental monitoring, and the like, it is expected to implement a sensor that quickly and easily detects a biological material such as bacteria.

For example, WO 2014/156584A discloses a sensor including a detection unit including a detection electrode and a polymer layer having a template with a three-dimensional structure complementary to a three-dimensional structure of a microorganism to be detected, and a crystal piece for detecting a mass change or the like in the detection unit. The template of the polymer layer has a three-dimensional structure complementary to the three-dimensional structure of the microorganism, and thus the template selectively traps a microorganism having the three-dimensional structure, but does not trap a microorganism that does not have the three-dimensional structure. Therefore, a target microorganism can be detected with high sensitivity based on a trapping state in the template.

In WO 2014/156584A, a polymer layer is formed using an actual microorganism. Specifically, WO 2014/156584A discloses a method for forming a polymer layer including a step of forming a polymer layer such that a microorganism is incorporated, and a step of destroying the incorporated microorganism. Accordingly, a template having a three-dimensional structure complementary to a three-dimensional structure of the actual microorganism is formed in the polymer layer.

In the sensor described in WO 2014/156584A, it is necessary to use the actual microorganism for forming the polymer layer having a template. However, some microorganisms are harmful to the human body, and it is necessary to pay close attention to handling. In the case of a microorganism that is difficult to obtain, it is difficult to stably produce a large number of templates. Therefore, it is a problem to implement a method for stably producing a polymer layer having a template while ensuring safety.

### SUMMARY

A replica of a biological material according to an application example of the present disclosure has a three-dimensional structure similar to a three-dimensional structure of a biological material, and a surface of the replica is made of an inorganic material.

A method for producing a replica of a biological material according to an application example of the present disclosure includes: a preparation step of preparing a template film having a template with a three-dimensional structure complementary to a three-dimensional structure of a biological material; a coating film forming step of forming a coating film made of an inorganic material on an inner surface of the template; a filling step of forming a replica having a three-dimensional structure similar to the three-dimensional structure of the biological material by supplying a filling material to the template on which the coating film is formed and solidifying the filling material; and a collection step of collecting the replica from the template film.

### BRIEF DESCRIPTION OF THE DRAWINGS

FIG. 1 is a schematic diagram showing a bacterium as an example of a biological material, and is a cross-sectional view of a replica of the biological material according to an embodiment.

FIG. 2 is an enlarged view of a portion A1 and a portion A2 in FIG. 1.

FIG. 3 is a partially enlarged view showing a modification of a body in FIG. 2.

FIG. 4 is a flowchart showing a method for producing the replica in FIG. 1.

FIG. 5 is a cross-sectional view illustrating the production method shown in FIG. 4, and is a schematic view showing the bacterium and a cross-sectional view of a template film having a template.

FIG. 6 is a cross-sectional view illustrating the production method shown in FIG. 4, and is an enlarged view of the portion A1 and a portion A3 in FIG. 5.

FIG. 7 is a cross-sectional view illustrating the production method shown in FIG. 4, and shows the template film in a state where a part of the bacterium is incorporated.

FIG. 8 is a cross-sectional view illustrating the production method shown in FIG. 4, and is an enlarged view of a portion A4 in FIG. 7.

FIG. 9 is a cross-sectional view illustrating the production method shown in FIG. 4, and is a cross-sectional view of the template film having the template.

FIG. 10 is a cross-sectional view illustrating the production method shown in FIG. 4 and showing a state where a coating film is formed on an inner surface of the template.

FIG. 11 is a cross-sectional view illustrating the production method shown in FIG. 4, and is an enlarged view of a portion A5 in FIG. 10.

FIG. 12 is a cross-sectional view illustrating the production method shown in FIG. 4 and showing a state where the body is formed inside the coating film.

FIG. 13 is a cross-sectional view illustrating the production method shown in FIG. 4 and showing a state where the template film is heated and expanded.

FIG. 14 is a cross-sectional view illustrating the production method shown in FIG. 4, and shows a state where the replica is attracted by a magnetic attraction force.

### DESCRIPTION OF EMBODIMENTS

Hereinafter, a replica of a biological material and a method for producing the replica of a biological material according to the present disclosure will be described in detail based on embodiments illustrated in the accompanying drawings.

### 1. Replica of Biological Material

First, a replica of a biological material according to an embodiment will be described.

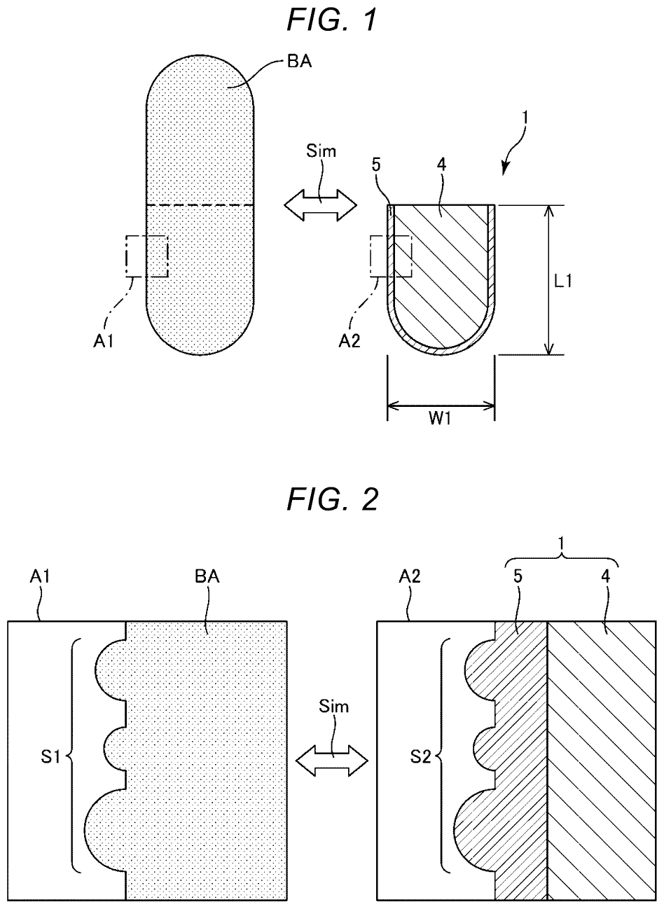

FIG. 1 is a schematic view showing a bacterium BA as an example of a biological material, and is a cross-sectional view of a replica 1 of the biological material according to the embodiment. FIG. 2 is an enlarged view of a portion A1 and a portion A2 in FIG. 1. In the following description, the replica 1 of the biological material is simply referred to as “replica 1”.

The bacterium BA shown in FIG. 1 is an example of the biological material and has a specific three-dimensional structure S1 as shown in FIG. 2 on a surface thereof. The three-dimensional structure S1 is derived from, for example, a specific molecular structure of the bacterium BA, and varies depending on the type of biological materials. On the other hand, the replica 1 shown in FIG. 1 has a similar structure S2 similar to the three-dimensional structure S1 of the bacterium BA.

The replica 1 is used for producing a polymer layer used for, for example, a biosensor. The polymer layer has a template having a three-dimensional structure complementary to the three-dimensional structure S1 of the bacterium BA, and has a property of selectively trapping the bacterium BA. Therefore, a biosensor or the like that detects the bacterium BA with high sensitivity can be implemented using the polymer layer.

The replica 1 can also be reused once or a plurality of times even after being used for producing the polymer layer. Accordingly, the polymer layer can be produced more efficiently by using the replica 1 as compared with a case of producing the polymer layer by using the actual bacterium BA.

Arrows Sim shown in FIGS. 1 and 2 indicate that the three-dimensional structure S1 of the bacterium BA and the similar structure S2 of the replica 1 are similar to each other. In the present specification, the term “similar” means that at least a part of the three-dimensional structure S1 of the bacterium BA and the similar structure S2 of the replica 1 have the same shape, or means a relation that the two have the same shape by enlarging or reducing either one of them. The same shape allows deviation in the shape due to a production error. When either one of them is enlarged or reduced, an enlargement ratio and a reduction ratio are as small as, for example, about 5% or less with respect to a substantially original size. Accordingly, it can be said that the replica 1 is a model obtained by imitating the bacterium BA in a substantially original size.

A part imitated by the replica 1 may be the entire bacterium BA or any part thereof. The replica 1 shown in FIG. 1 is obtained by imitating a part of the rod-shaped bacterium BA in a longitudinal direction. In addition, the biological material is not limited to a bacterium, and may be, for example, a microorganism other than a bacterium, such as a virus, a fungus, and a protozoan, a cell, or an exosome.

Specific examples of the bacterium include Escherichia coli, Pseudomonas aeruginosa, Staphylococcus aureus, and Bacillus subtilis. Examples of the virus include a hepatitis A virus, an adenovirus, a rotavirus, and a norovirus. Examples of the fungus include Candida. Examples of the protozoan include Cryptosporidium.

The replica 1 shown in FIG. 2 has a core-shell structure including a body 4 and a coating film 5 that covers a surface of the body 4. Therefore, the similar structure S2 is a part obtained by forming a part of the coating film 5 into a three-dimensional shape. The coating film 5 is made of an inorganic material. The inorganic material is superior to an organic material in mechanical properties such as mechanical strength, rigidity, and wear resistance, and chemical properties such as corrosion resistance and heat resistance. Therefore, when the coating film 5 is made of the inorganic material, the replica 1 in which the similar structure S2 is stably maintained can be obtained.

When the longest axis that can be taken by the replica 1 is defined as a major axis, a length L1 of the major axis varies depending on the type of the biological materials. The length L1 is, for example, 0.3 μm or more and 10 μm or less in the case of a bacterium. When an axis perpendicular to the major axis is defined as a minor axis, a length W1 of the minor axis is, for example, 0.1 μm or more and 5 μm or less in the case of a bacterium.

### 1.1. Body

The body 4 supports the coating film 5 having the similar structure S2. A material of the body 4 may be the same as or different from a material of the coating film 5.

In the former case, the adhesion between the body 4 and the coating film 5 can be enhanced. Accordingly, the replica 1 having high mechanical strength can be obtained. In this case, the body 4 and the coating film 5 may be integrated with each other, and boundaries may not be distinguished from each other. In this case, at least the surface may also be made of an inorganic material.

In the latter case, properties different from each other depending on materials are imparted to the body 4 and the coating film 5. Accordingly, for example, the replica 1 which has the similar structure S2 having high reproducibility with respect to the three-dimensional structure S1 and which has high production easiness can be obtained.

Examples of the material of the body 4 include organic materials such as resins, and inorganic materials such as metals, metal-based compounds, non-metals, and non-metal-based compounds. A composite material obtained by combining an organic material and an inorganic material may be used.

Examples of the metals include a simple substance of a metallic element such as Fe, Co, Ni, Cu, Ti, Al, Mg, Ag, Au, Pt, Mo, W, Nb, and Ta, and an alloy containing the metallic element as a main component. Examples of the metal-based compound include metal oxides, metal carbides, metal nitrides, metal chlorides, metal sulfides, metal carbonates, and metal hydroxides.

Examples of the non-metals include S1, B, and C. Examples of the non-metal-based compounds include non-metal oxides, non-metal carbides, and non-metal nitrides.

The material of the body 4 may be a composite material obtained by combining two or more kinds selected from the above-described inorganic materials, or may be a composite material obtained by combining at least one kind selected from the above-described inorganic materials and another inorganic material.

Among them, the material of the body 4 preferably includes a metal or a metal-based compound. The body 4 can favorably reinforce the coating film 5 for a long period of time because the metal or the metal-based compound has particularly high mechanical properties and chemical properties. A content of the metal or metal-based compound for obtaining such an effect is, for example, 80 mass % or more.

When the material of the body 4 does not include a metal or a metal-based compound and is a resin, examples thereof include a thermoplastic resin, a thermosetting resin, and a photocurable resin. When the material of the body 4 includes a resin, the production easiness of the replica 1 can be enhanced. A content of the resin for obtaining such an effect is, for example, 80 mass % or more.

In particular, when a thermosetting resin or a photocurable resin is used, the rigidity and heat resistance of the body 4 can be increased as compared with the case of using a thermoplastic resin. A timing of curing can be controlled by applying heat to the thermosetting resin or by emitting light to the photocurable resin. Accordingly, the replica 1 having high production easiness can be obtained. In the case of the photocurable resin, the resin is polymerized by light irradiation, and thus a volume change in the resin due to curing is small, and the shape and size of the original template can be maintained with higher accuracy for the replica 1.

Examples of the thermoplastic resin include polyvinyl acetates, polyvinyl alcohols, polyvinyl butyrals, polystyrenes, an acrylonitrile butadiene styrene (ABS) resin, a methacrylic resin, a noryl resin, polyurethanes, an ionomer resin, cellulose-based plastic, polyethylenes, polypropylenes, polyamides, polycarbonates, polyacetals, polyphenylene sulfides, polyvinylidene chlorides, polyesters, and a fluororesin. One kind of them may be used alone, or two or more kinds thereof may be used in combination.

Examples of the thermosetting resin and the photocurable resin include polyimides, an epoxy resin, a phenol resin, a urea resin, a melamine resin, a silicone resin, polyamide imides, benzocyclobutenes, benzoxazines, and a cyanate resin. One kind of them may be used alone, or two or more kinds thereof may be used in combination.

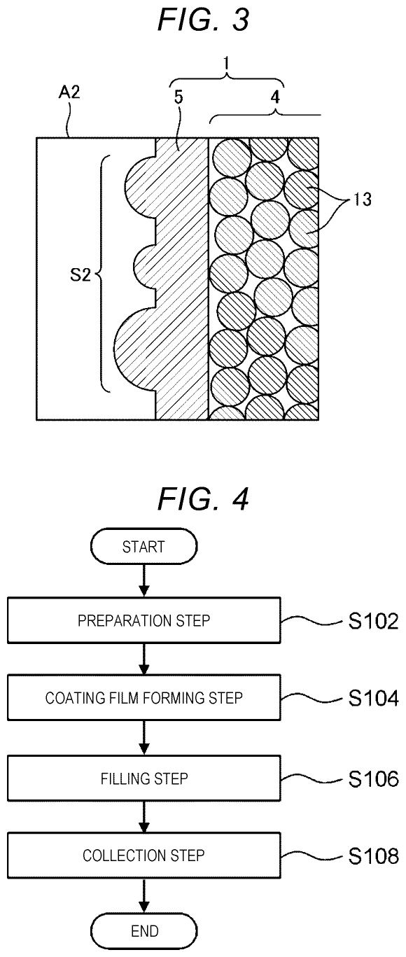

FIG. 3 is a partially enlarged view showing a modification of the body 4 in FIG. 2.

The body 4 shown in FIG. 3 includes an aggregate of particles 13. When forming the aggregate, the particles 13 are fixed to or bonded to each other to support the coating film 5. When the particles 13 are used, fluidity and formability can be imparted to the material. A material that is difficult to fill can also be used as the material of the body 4 when the material is formed into particles.

Examples of a material of the particles 13 include the above-described organic materials and inorganic materials. A magnetic material is preferably used as the material of the particles 13. When a magnetic field is applied to the particles 13, a magnetic attraction force can be generated in the particles 13 by using a magnetic material. A method for producing the replica 1 to be described later can be efficiently performed by using the magnetic attraction force.

The magnetic material may be a paramagnetic material, and is preferably a ferromagnetic material. The ferromagnetic material has a high permeability, and thus a large magnetic attraction force can be generated in response to the applied magnetic field.

The ferromagnetic material may be a hard magnetic material, and is preferably a soft magnetic material. The soft magnetic material has a high permeability and a low coercivity. Therefore, a large magnetic attraction force is generated in a state where the magnetic field is applied, and on the other hand, the magnetic attraction force can be greatly reduced when the application of the magnetic field is stopped. Accordingly, the method for producing the replica 1 to be described later can be performed particularly efficiently by using on/off of the application of the magnetic field. The coercivity of the soft magnetic material is preferably 800 A/m or less, and more preferably 400 A/m or less.

Examples of the soft magnetic material include metal-based soft magnetic materials such as an Fe-based alloy, a Ni-based alloy, and a Co-based alloy, and oxide-based soft magnetic materials such as a spinel ferrite, a magnetoplumbite ferrite, and a garnet ferrite which all contain Fe₃O₄ as a main component. Among them, an oxide-based soft magnetic material is preferably used. The oxide-based soft magnetic material is useful as the material of the particles 13 because particles of a nm-size can be produced at low cost.

An average particle diameter of the particles 13 is preferably 5 nm or more and 1 μm or less, more preferably 10 nm or more and 300 nm or less, and still more preferably 20 nm or more and 200 nm or less. When the average particle diameter of the particles 13 is within the above range, a filling property of the particles 13 can be enhanced, and a dense body 4 can be obtained. When the average particle diameter of the particles 13 is less than the lower limit, handling of the particles 13 becomes difficult, and aggregation of the particles 13 may occur. In the case of a magnetic material, a coercivity sufficient for induction to a template portion to be described later may not be obtained. On the other hand, when the average particle diameter of the particles 13 exceeds the upper limit, gaps between the particles 13 become large, and the denseness of the body 4 may decrease.

The average particle diameter of the particles 13 is determined, for example, by observing the cross section of the body 4 with a scanning electron microscope (SEM), measuring particle diameters of 10 or more particles 13 freely selected, and calculating an average value thereof.

When the particles 13 are used, a binding agent that binds the particles 13 to each other may be used as necessary. As the binding agent, for example, the above-described resin is used.

In addition, any additive may be added to the material of the body 4.

### 1.2. Coating Film

The coating film 5 is made of an inorganic material as described above. Examples of the inorganic material include oxides, nitrides, carbides, sulfides, and fluorides. Among them, the inorganic material is preferably an oxide or a nitride. The oxide and the nitride are particularly excellent in mechanical properties and chemical properties among the inorganic materials. Therefore, the coating film 5 capable of particularly stably maintaining the similar structure S2 can be obtained.

Examples of the oxide include silicon oxides, titanium oxides, aluminum oxides, zirconium oxides, hafnium oxides, niobium oxides, zinc oxides, tin oxides, yttrium oxides, magnesium oxides, calcium oxides, and boron oxides.

Among them, silicon oxides, titanium oxides, and aluminum oxides are preferably used. These oxides can form the dense coating film 5, and are particularly good in mechanical properties and chemical properties. The silicon oxides are oxides represented by a composition formula SiOₓ (0<x≤2), and are preferably SiO₂. The titanium oxides are oxides represented by a composition formula TiOₓ (0<x≤2), and are preferably TiO₂. The aluminum oxides are oxides represented by a composition formula AlOₓ (0<x≤2), and are preferably Al₂O₃.

Examples of the nitrides include gallium nitrides, silicon nitrides, aluminum nitrides, boron nitrides, and titanium nitrides.

The material of the coating film 5 may be a composite material obtained by combining two or more kinds selected from the above-described inorganic materials, or a composite material obtained by combining at least one kind selected from the above-described inorganic materials and another inorganic material. Examples of the composite material include an Al₂O₃—SiO₂ composite material.

An average thickness of the coating film 5 is not particularly limited, and is preferably 1 nm or more and 500 nm or less, more preferably 3 nm or more and 100 nm or less, and still more preferably 5 nm or more and 50 nm or less. When the average thickness of the coating film 5 is within the above range, a thickness sufficient for forming the similar structure S2 can be ensured, and an adverse effect caused by the coating film 5 being too thick can be prevented. That is, when the average thickness of the coating film 5 is less than the lower limit, there is a possibility that the similar structure S2 having various protrusion heights cannot be sufficiently formed. On the other hand, when the average thickness of the coating film 5 exceeds the upper limit, it takes more time to form the coating film 5, and thus the production efficiency of the replica 1 may decrease.

The average thickness of the coating film 5 is determined by observing a cross section of the replica 1 with a scanning electron microscope (SEM) or a scanning transmission electron microscope (STEM), measuring thicknesses of 10 or more positions freely selected, and calculating an average value thereof.

### 2. Method for Producing Replica of Biological Material

Next, a method for producing the replica of the biological material according to the embodiment will be described. In the following description, the method for producing the above-described replica 1 will be described as an example.

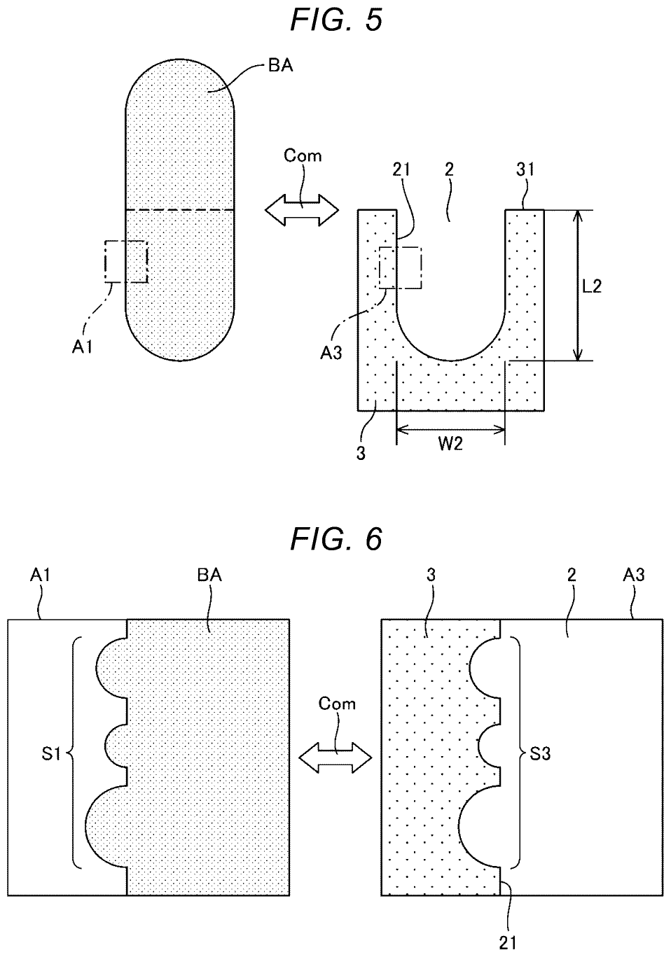

FIG. 4 is a flowchart showing the method for producing the replica 1 in FIG. 1. FIGS. 5 to 14 are cross-sectional views illustrating the production method shown in FIG. 4. In FIGS. 5 to 14, the same configurations as those in FIGS. 1 to 3 are denoted by the same reference signs.

The method for producing the replica 1 shown in FIG. 4 includes a preparation step S102, a coating film forming step S104, a filling step S106, and a collection step S108.

### 2.1. Preparation Step

In the preparation step S102, a template film 3 is prepared. The template film 3 has a template 2 with a complementary structure S3 complementary to the three-dimensional structure S1 of the bacterium BA. The template 2 is a hole formed by recessing an upper surface 31 of the template film 3, and the complementary structure S3 is provided on an inner surface 21 of the template 2.

FIG. 5 is a schematic view showing the bacterium BA, and is a cross-sectional view of the template film 3 having the template 2. FIG. 6 is an enlarged view of the portion A1 and a portion A3 in FIG. 5.

Arrows Com shown in FIGS. 5 and 6 indicate that the three-dimensional structure S1 of the bacterium BA and the complementary structure S3 of the template 2 are complementary to each other. In the present specification, the term “complementary” means that at least a part of the three-dimensional structure S1 of the bacterium BA and the complementary structure S3 of the template 2 are combined with each other in shape without gaps, or means a relation that the two are combined with each other in shape without gaps by enlarging or reducing either one of them. The shapes to be combined allow deviation in the shapes due to production errors (gaps due to production errors). When either one of them is enlarged or reduced, an enlargement ratio and a reduction ratio are as small as, for example, about 5% or less with respect to an original size. Accordingly, it can be said that the template 2 is a model obtained by transferring the bacterium BA in a substantially original size.

The site to be transferred to the template 2 may be the entire bacterium BA or any part thereof. The template 2 shown in FIG. 5 is obtained by transferring a part of the rod-shaped bacterium BA in the longitudinal direction. In addition, the biological material is not limited to a bacterium, and may be, for example, a microorganism other than a bacterium, such as a virus, a fungus, and a protozoan, a cell, or an exosome.

When the longest axis that can be taken by the template 2 is defined as a major axis, a length L2 of the major axis varies depending on the type of biological materials. For example, in the case of a bacterium, the length L2 is 0.3 μm or more and 10 μm or less. When an axis perpendicular to the major axis is defined as a minor axis, a length W2 of the minor axis is, for example, 0.1 μm or more and 5 μm or less in the case of a bacterium.

The template film 3 having the template 2 is formed, for example, as follows.

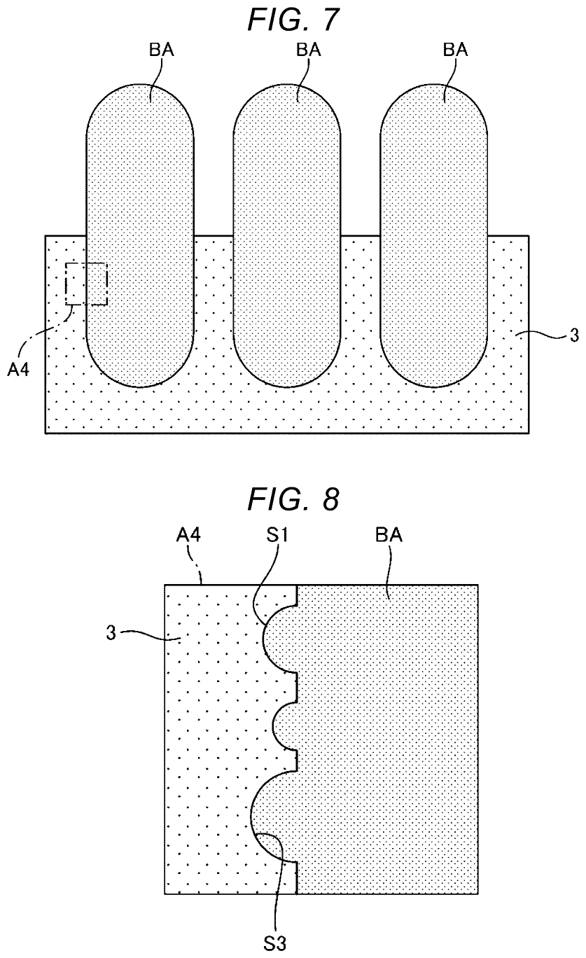

First, a solution containing monomers and the bacteria BA is prepared, and the solution is applied onto a base material to form a liquid film. Next, an electric field is applied to the liquid film. The bacteria BA are generally negatively charged. Therefore, the bacteria BA can be migrated to a base material side by appropriately setting a direction of the electric field. At this timing, a polymerization reaction is caused in the monomers in the solution applied on the base material. Accordingly, as shown in FIGS. 7 and 8, the template film 3 in which a part of the bacteria BA is incorporated is obtained. FIG. 8 is an enlarged view of a portion A4 in FIG. 7.

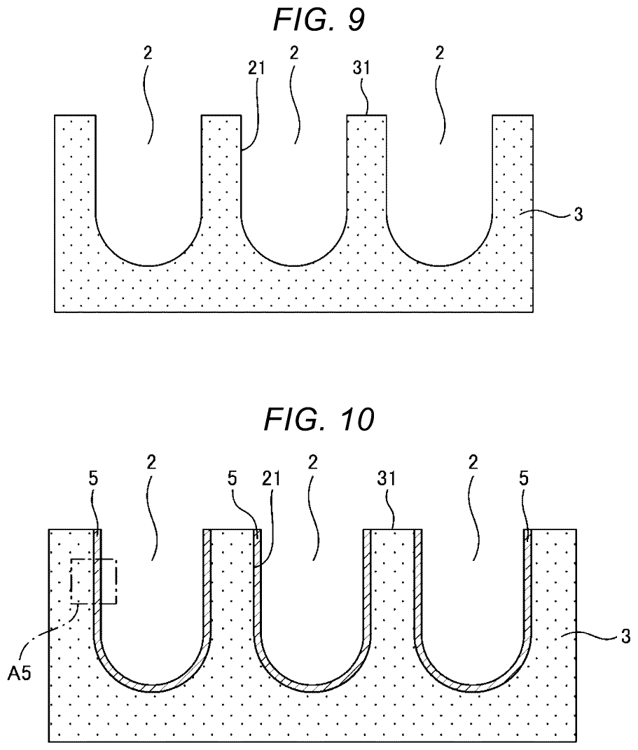

Next, the bacteria BA are removed. Accordingly, as shown in FIG. 9, the template film 3 having the templates 2 is obtained. As a method for removing the bacteria BA, for example, a method for bringing the bacteria BA into contact with a solution containing a lytic enzyme is used.

The monomers are not particularly limited, and examples thereof include pyrrole, aniline, thiophene, and derivatives thereof. For example, when pyrrole is used as the monomer, the template film 3 becomes a polypyrrole film. In this case, examples of the polymerization reaction include an electrolytic polymerization reaction, and the polymerization reaction in this step is not limited thereto.

### 2.2. Coating Film Forming Step

In the coating film forming step S104, as shown in FIG. 10, the coating film 5 is formed on the inner surface 21 of the template 2. The coating film 5 is made of an inorganic material, and is formed in a state of being formed according to the complementary structure S3 of the template 2 as shown in FIG. 11. Accordingly, a three-dimensional structure formed by transferring the complementary structure S3 to the coating film 5 is obtained. The three-dimensional structure has a similar relation with the three-dimensional structure S1 of the bacterium BA. Accordingly, the coating film 5 having the above-described similar structure S2 is obtained. FIG. 11 is an enlarged view of a portion A5 in FIG. 10.

The coating film 5 may be formed by a liquid-phase deposition method, and is preferably formed by a vapor-phase deposition method, and more preferably formed by an atomic layer deposition (ALD) method. In the ALD method, a raw material can be deposited at an atomic layer level, and thus the coating film 5 that is dense and thin can be formed. In addition, in the ALD method, a raw material, an oxidant, or the like can flow around to a recessed part or a shaded part. Therefore, by using the ALD method, the coating film 5 having the similar structure S2 obtained by more reliably transferring the complementary structure S3 of the template 2 can be obtained. Therefore, the similar structure S2 is a more faithful imitation of the three-dimensional structure S1 of the bacterium BA.

Hereinafter, as an example, a method for forming the coating film 5 made of a silicon oxide by the ALD method will be described. First, the template film 3 having the templates 2 shown in FIG. 9 is charged into a chamber in which evacuation and atmosphere control can be performed. Next, a precursor serving as a raw material of the coating film 5 is charged into the chamber, and is adsorbed to the inner surface 21 of the template 2 including the complementary structure S3. The precursor is appropriately selected according to the inorganic material constituting the coating film 5. When the inorganic material is a silicon oxide, examples of the precursor include dimethylaminosilane, methylethylaminosilane, diethylaminosilane, tris(dimethylamino) silane, bis(diethylamino) silane, and bis(tert-butylamino)silane. Next, after the excess precursor is discharged, an oxidant is introduced into the chamber. Examples of the oxidant include ozone, plasma oxygen, and water vapor. The oxidant reacts with the precursor adsorbed to the template 2 to form the coating film 5.

The treatment temperature in the ALD method is appropriately set according to the type of precursors or the like, and is preferably 30° C. or higher and 150° C. or lower, and more preferably 30° C. or higher and 100° C. or lower. Such a temperature range is a relatively low temperature in the vapor-phase film deposition method, so that the coating film 5 can be formed while preventing thermal denaturation of the template film 3. As a result, finally, the coating film 5 having the similar structure S2 obtained by particularly faithfully imitating the three-dimensional structure S1 of the bacterium BA is obtained. For the film formation in such a temperature range, for example, a room-temperature ALD method using a room-temperature ALD film formation device is used.

### 2.3. Filling Step

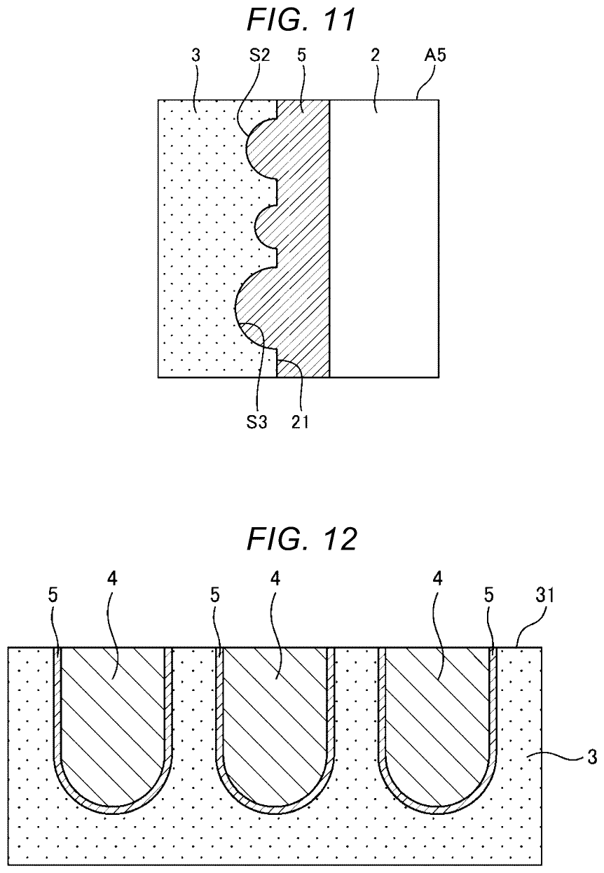

In the filling step S106, a filling material is supplied to the template 2 and solidified. Accordingly, the bodys 4 shown in FIG. 12 are obtained. The filling material is not particularly limited as long as it is a material by which the bodys 4 can be obtained by solidification. The term “solidification” as used in the present specification means that a material having fluidity and formability is solidified after formation to maintain a shape thereof, and also includes curing.

Examples of the filling material include raw materials of the above-described materials of the body 4. Specific examples of the raw materials include a solution containing a resin before solidification, a dispersion containing a resin powder, a precursor of an inorganic material, an inorganic material powder, and a magnetic material powder.

The filling material is prepared in a state of having fluidity and formability and is supplied to the template 2. When the filling material overflows from the template 2, the surplus may be removed using a squeegee as necessary. Next, the filling material is solidified by applying heat or light as necessary. Accordingly, the body 4 is obtained. Then, the replica 1 including the body 4 and the coating film 5 is obtained.

If necessary, a binding agent (binder), a dispersing agent, a flame retardant, an ultraviolet absorbing agent, a filler, an antioxidant, and the like may be added to the filling material.

When the filling material overflowing from the template 2 is not removed, a plurality of individuals are solidified in a connected state. Such a state is also referred to as a “replica complex” in the present specification, and is a modification of the replica 1 according to the embodiment.

The replica complex is formed by connecting a plurality of individuals. With the state of the replica complex, the collection step S108 to be described later is easier, and the production process is efficient. The individual is a part having the same configuration as the above-described replica 1, that is, a part including the coating film 5 having the similar structure S2 having a similar relation with the three-dimensional structure S1 of the bacterium BA. The replica complex collected in the collection step S108 may be supplied to the step of separating individuals or may be used as it is.

In the present step, a support substrate may be attached to the replica complex. Accordingly, the mechanical properties of the replica complex can be enhanced. In this case, in the collection step S108 to be described later, an operation of separating the replica complex from the template film 3 can be performed for each support substrate. Accordingly, the collection step S108 can be performed more efficiently.

### 2.4. Collection Step

In the collection step S108, the replica 1 is collected from the template film 3. In this collection, the replica 1 may be separated from the template film 3. Accordingly, the replica 1 can be collected in a state where the similar structure S2 of the coating film 5 is favorably maintained.

Examples of the method for collecting the replica 1 include a method for performing an operation of heating the template film 3, a method for performing an operation of pulling out the replica 1 from the template film 3, and a method for performing an operation of removing the template film 3.

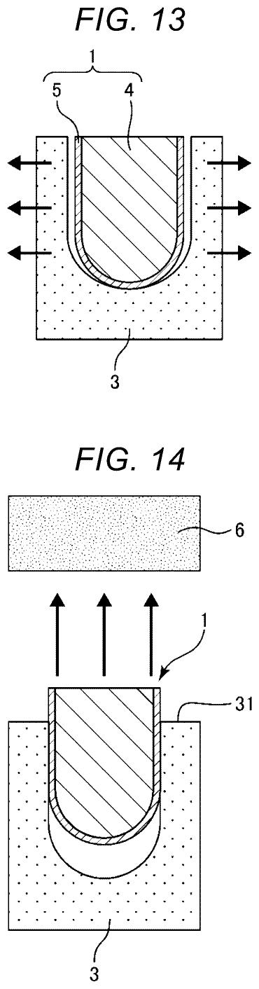

In the operation of heating the template film 3, the template film 3 is expanded by heating. Accordingly, as shown in FIG. 13, a gap can be formed between the template film 3 and the replica 1. As a result, the replica 1 can be smoothly separated and collected. The heating temperature is not particularly limited as long as it is a temperature at which the template film 3 is not thermally denatured, and the heating temperature is, for example, 40° C. or higher and 100° C. or lower. When the body 4 of the replica 1 is made of an inorganic material, the replica 1 can be separated even by heating at a lower temperature because a difference in thermal expansion between the replica 1 and the template film 3 is large.

The operation of pulling out the replica 1 from the template film 3 is effective when the body 4 of the replica 1 contains a magnetic material, for example. In this case, when a magnetic field is applied to the replica 1, a magnetic attraction force is generated at the replica 1. Specifically, as shown in FIG. 14, when a magnetic field generation source 6 is brought close to an upper surface 31 side of the template film 3, a magnetic attraction force toward the magnetic field generation source 6 is generated at the replica 1. Accordingly, the replica 1 can be collected more easily. Examples of the magnetic field generation source 6 include a permanent magnet and an electromagnet. Among them, the electromagnet is preferably used from the viewpoint that the collected replica 1 can be easily separated from the magnetic field generation source 6.

In the operation of removing the template film 3, the template film 3 is removed by an ashing treatment or a wet treatment. Specific examples of the ashing treatment include plasma ashing and ozone ashing. In the plasma ashing, the template film 3 is ashed and removed by a plasma treatment. In the ozone ashing, ozone is brought into contact with the template film 3 to decompose and vaporize the template film 3. In the wet treatment, the template film 3 is dissolved and removed.

Among them, a method using the plasma treatment is preferable. When the plasma treatment is used, the template film 3 can be removed while minimizing the influence on the replica 1. As the plasma treatment, for example, oxygen plasma is preferably used. The replica 1 includes the coating film 5 made of an inorganic material, and thus the replica 1 has good resistance to the plasma treatment.

### 3. Method for Using Replica

The replica 1 has a similar structure S2 similar to the three-dimensional structure S1 of the bacterium BA. Therefore, the replica 1 is a pattern for forming the template 2 instead of the actual bacterium BA. That is, in the above-described method for forming the template film 3 having the template 2, the bacterium BA can be replaced with the replica 1. When the replica 1 is charged, a polymer layer equivalent to the above-described template film 3 by inducing the replica 1 through electrophoresis as in the case of the bacterium BA can be formed. On the other hand, when the replica 1 is not charged, magnetism originating from the particles 13 can be used instead. Then, a polymer layer equivalent to the above-described template film 3 can be produced by inducing the replica 1 through an electromagnet using a magnetic force instead of electrophoresis. The polymer layer thus produced can be used in, for example, a biosensor for detecting a biological material.

When the replica 1 is used, the polymer layer can be produced without using the actual bacterium BA. That is, once the replica 1 is produced, it is not necessary to handle the bacterium BA harmful to the human body, and thus safety can be ensured. In addition, the replica 1 can be repeatedly used as a pattern replacing the bacterium BA. Therefore, the polymer layer can be efficiently produced, and the cost can be easily reduced. Even in the case of the bacterium BA that is difficult to obtain, a polymer layer can be stably produced according to the demand.

### 3. Effects of Embodiment

As described above, the replica 1 of the biological material according to the embodiment has the similar structure S2 (similar three-dimensional structure) similar to the three-dimensional structure S1 of the bacterium BA (biological material), and the surface of the replica 1 is made of an inorganic material.

According to the replica 1, for example, when a polymer layer used for a biosensor or the like is produced, the polymer layer can be formed without using the actual bacterium BA. In addition, even when producing a polymer layer applied to the bacterium BA that is difficult to obtain, the polymer layer can be stably produced by producing the replica 1 in advance.

The inorganic material is preferably an oxide or a nitride. The oxide and the nitride are particularly excellent in mechanical properties and chemical properties among inorganic materials. Therefore, the replica 1 capable of particularly stably maintaining the similar structure S2 can be obtained.

The replica 1 preferably includes the body 4 and the coating film 5. The coating film 5 covers the surface of the body 4 and is made of an inorganic material.

According to such a configuration, when materials of the body 4 and the coating film 5 are selected, properties suitable for both can be imparted.

The material of the body 4 may be the same as the material of the coating film 5. In this case, the adhesion between the body 4 and the coating film 5 can be enhanced. Accordingly, the replica 1 having high mechanical strength can be obtained.

The material of the body 4 may be different from the material of the coating film 5. In this case, different properties depending on the material can be imparted. Accordingly, for example, the replica 1 which has the similar structure S2 having high reproducibility with respect to the three-dimensional structure S1 and has high production easiness can be obtained.

The material of the body 4 may include a metal or a metal-based compound. The body 4 can favorably reinforce the coating film 5 for a long period of time because the metal or the metal-based compound has particularly high mechanical properties and chemical properties.

The material of the body 4 may include a resin. Accordingly, the production easiness of the replica 1 can be enhanced.

The body 4 may contain an aggregate of the particles 13. When the particles 13 are used, fluidity and formability can be imparted to the material. Accordingly, a material that is difficult to fill can also be used as a material of the body 4.

The particles 13 may be made of a magnetic material. When a magnetic field is applied to the particles 13, a magnetic attraction force can be generated in the body 4 including the particles 13 by using a magnetic material. Therefore, the replica 1 that can be efficiently produced can be obtained.

The replica 1 may be a complex which includes a plurality of parts (individuals) having the similar structure S2 (similar three-dimensional structure), and is formed by connecting the parts to each other.

With the replica complex in which the individuals are connected to each other, the collection process is easier than the case of collecting the respective individuals. Therefore, a replica that can be produced efficiently can be obtained.

The method for producing the replica 1 of the biological material according to the embodiment includes the preparation step S102, the coating film forming step S104, the filling step S106, and the collection step S108. In the preparation step S102, the template film 3 having the template 2 with the complementary structure S3 (complementary three-dimensional structure) complementary to the three-dimensional structure S1 of the bacterium BA (biological material) is prepared. In the coating film forming step S104, the coating film 5 made of an inorganic material is formed on the inner surface 21 of the template 2. In the filling step S106, the replica 1 having the similar structure S2 (similar three-dimensional structure) similar to the three-dimensional structure S1 of the bacterium BA is formed by supplying the filling material to the template 2 on which the coating film 5 is formed and solidifying the filling material. In the collection step S108, the replica 1 is collected from the template film 3.

According to such a configuration, the replica 1 obtained by imitating the bacterium BA (biological material) in a substantially original size can be produced. Regarding the replica 1, for example, when a polymer layer used for a biosensor or the like is produced, the polymer layer can be formed without using the actual bacterium BA. In addition, even when producing a polymer layer applied to the bacterium BA that is difficult to obtain, the polymer layer can be stably produced by producing the replica 1 in advance.

The method for producing the replica 1 of the biological material may further include a complex forming step. In the complex forming step, the complex which includes a plurality of parts (individuals) having the similar structure S2 (similar three-dimensional structure) and is formed by connecting the parts to each other is formed.

According to such a configuration, the collection step S108 is performed easier than the case of collecting respective individuals, and the replica producing process is more efficient.

The complex forming step may include an operation of attaching the support substrate to the complex, and the collection step S108 may include an operation of collecting the complex supported by the support substrate from the template film 3.

According to such a configuration, the collection step S108 can be performed more efficiently.

The coating film forming step S104 preferably includes an operation of forming the coating film by the room-temperature ALD method. Accordingly, the coating film 5 can be formed while preventing the thermal denaturation of the template film 3. As a result, finally, the coating film 5 having the similar structure S2 obtained by particularly faithfully imitating the three-dimensional structure S1 of the bacterium BA is obtained.

The collection step S108 may include an operation of heating and expanding the template film 3. Accordingly, a gap can be formed between the template film 3 and the replica 1. As a result, the replica 1 can be smoothly separated and collected.

The filling material may be a magnetic material. In this case, the collection step S108 preferably includes an operation of applying a magnetic field to the replica 1 of the bacterium BA (biological material). Accordingly, a magnetic attraction force toward the magnetic field generation source 6 is generated at the replica 1. As a result, the replica 1 can be collected more easily.

The collection step S108 may include an operation of removing the template film 3 by the plasma treatment. Accordingly, the template film 3 can be removed while minimizing the influence on the replica 1.

Although the replica of the biological material and the method for producing the replica of the biological material of the present disclosure were described based on the illustrated embodiment, the present disclosure is not limited thereto. For example, in the replica of the biological material of the present disclosure, a configuration of each portion of the embodiment may be replaced with any configuration having the same function, and any other components may be added to the embodiment. In addition, the method for producing the replica of the biological material of the present disclosure may be a method in which a step for any purpose is added to the embodiment.

What is claimed is:

1. A replica of a biological material, the replica having a three-dimensional structure similar to a three-dimensional structure of the biological material and having a surface made of an inorganic material.

2. The replica of a biological material according to claim 1, wherein
the inorganic material is an oxide or a nitride.

3. The replica of a biological material according to claim 1, comprising:
a body; and
a coating film configured to cover a surface of the body and made of the inorganic material.

4. The replica of a biological material according to claim 3, wherein
a material of the body is same as a material of the coating film.

5. The replica of a biological material according to claim 3, wherein
a material of the body is different from a material of the coating film.

6. The replica of a biological material according to claim 4, wherein
the material of the body includes a metal or a metal-based compound.

7. The replica of a biological material according to claim 5, wherein
the material of the body includes a resin.

8. The replica of a biological material according to claim 3, wherein
the body contains an aggregate of particles.

9. The replica of a biological material according to claim 8, wherein
the particles are made of a magnetic material.

10. The replica of a biological material according to claim 1, which is a complex including a plurality of parts having three-dimensional structures similar to the three-dimensional structure and formed by connecting the parts to each other.

11. A method for producing a replica of a biological material, the method comprising:
a preparation step of preparing a template film having a template having a three-dimensional structure complementary to a three-dimensional structure of a biological material;
a coating film forming step of forming a coating film made of an inorganic material on an inner surface of the template;
a filling step of forming a replica having a three-dimensional structure similar to the three-dimensional structure of the biological material by supplying a filling material to the template on which the coating film is formed and by solidifying the filling material; and
a collection step of collecting the replica from the template film.

12. The method for producing a replica of a biological material according to claim 11, further comprising:
a complex forming step of forming a complex which includes a plurality of parts having three-dimensional structures similar to the three-dimensional structure and which is formed by connecting the parts to each other.

13. The method for producing a replica of a biological material according to claim 12, wherein
the complex forming step includes an operation of attaching the complex to a support substrate, and
the collection step includes an operation of collecting the complex supported by the support substrate from the template film.

14. The method for producing a replica of a biological material according to claim 11, wherein
the coating film forming step includes an operation of forming the coating film by a room-temperature ALD method.

15. The method for producing a replica of a biological material according to claim 11, wherein
the collection step includes an operation of heating and expanding the template film.

16. The method for producing a replica of a biological material according to claim 11, wherein
the filling material is a magnetic material, and
the collection step includes an operation of applying a magnetic field to the replica of the biological material.

17. The method for producing a replica of a biological material according to claim 11, wherein
the collection step includes an operation of removing the template film by a plasma treatment.
